(19) United States
(12) Patent Application Publication
(10) Pub. No.: US 2015/0044748 A1
(43) Pub. Date: Feb. 12, 2015

(54) Method for Processing Plant Remains

(71) Applicant: Bühle Barth GmbH, Freiberg (DE)

(72) Inventor: Tobias Lohmüller, Freiberg (DE)

(21) Appl. No.: 14/375,974

(22) PCT Filed: Feb. 4, 2013
(86) PCT No.: PCT/EP2013/052137

§ 371 Date: Jul. 31, 2014

(30) Foreign Application Priority Data
Feb. 2, 2012 (EP) 12153570.2

(51) Int. Cl. A23L 1/308; C12P 5/02; A23L 2/52

(52) U.S. Cl. CPC A23L 1/308; A23L 2/52; C12P 5/023; A23V 2002/00
USPC 435/167; 426/49; 426/615

(57) ABSTRACT

The invention relates to a method and to a system for processing plant remains, in particular shells of seeds and nuts, even more in particular shells of cocoa beans, shells of grain seeds, and rice remains. The method comprises the following steps: (i) providing plant remains having a shell portion of at least 20 wt %; and (ii) at least partially hydrolyzing constituents of the plant remains, in particular at least partially hydrolyzing and/or fermenting a carbohydrate, a fat, and/or a protein. A liquid phase having dissolved constituents and a solid phase can subsequently be separated. The solid portion can be used as dietary fiber and the liquid phase can be used as feed for a biogas plant.

The invention relates to a method and a system for processing plant remains, in particular shells of seeds and nuts, even more in particular shells of cocoa beans, shells of grain seed and rice remains.

Many proposals are known from the prior art for adding indigestible ballast substances such as bran, carboxymethylcellulose, pectin-coated cellulose, lignin, hemicelluloses, pentosans, gum and pectins to foods and confectionery.

Edible fibers from cocoa shells and other plant constituents such as grain shells, remains of rice grains, germs and/or nuts have also been recognized as a possible food constituent e.g. on account of their cellulose content.

The outer covering of cocoa beans is referred to as cocoa shell, also called testa. After harvesting, the cocoa fruits are fermented on average for six days, during which the fruit flesh detaches from the beans and the beans develop their desired taste and the brown coloration. Then, the beans are dried, cleaned and roasted. The shells of the beans are broken and separated from the beans.

The cocoa shells are generally considered to be a waste product. However, the cocoa shells also contain valuable ingredients such as e.g. polyphenols (1 to 2%), alkaloids such as theobromine (1 to 2%), vitamins such as vitamin D, minerals, amino acids and soluble as well as insoluble dietary fibers. The cocoa shells still contain up to 6% fat.

The use of cocoa shells in foods is restricted by law in many areas. Although a maximum amount is no longer defined since implementation of the EU Guideline 2000/36/EC (2000) e.g. in Germany, manufacturers and consumers are nevertheless still interested in monitoring the shell fraction since these can contain substances that are harmful to health such as pesticides, microorganisms, mycotoxins, free fatty acids (FFA) and heavy metals, and the shells can cause damage to the roller mills.

Moreover, cocoa shell fibers in particular for the most part have swelling and/or gellifying properties. They can bind fat and water and therefore influence the viscosity of the mixture to which they are added. The cocoa shell fibers have therefore hitherto only been suitable for use in a few specific foods. The use of cocoa shell fibers in an acidified food product such as e.g. a cheese is shown for example in EP 2 174 555.

Moreover, it is known to use cocoa shells in aromatic drinks, cocoa products, mulch, fertilizers and animal feed.

The production of edible fibers from cocoa shells is shown for example in EP 0 068 229, where the shells of the cocoa beans are subjected to a wet cleaning, dried and ground. The finely ground end product is reportedly added to foods and confectionery to improve the digestion-promoting properties.

The mechanical comminution of cocoa bean shells is known from EP 1 733 624. The document shows a method for grinding cocoa shells, where the cocoa shells are entrained in a fluidizing processing device, also called “jet mill”, in the introduced air, and ground. Other methods such as pulverizing in an air-swept mill are likewise possible, but lead to greater wear and tear.

The use of cocoa shell extracts is likewise known. A food dye can be obtained for example according to U.S. Pat. No. 4,156,030 by extraction with an acidic ethanol solution.

According to U.S. Pat. No. 4,532,147, pigments can also be extracted with the help of an aqueous alcohol.

A chocolate flavor can be provided as a result of treating cocoa shells with an alkalizing agent, as shown in EP 2 174 557.

The known methods for treating chocolate shells for providing food additives modify in particular the environment of the cocoa shell fibers, but leave the composition essentially unchanged. Targeted removal of undesired constituents does not take place. In particular, the slimes responsible for the swelling and/or gellifying properties contain proteins and sugars which are not removed by conventional technologies such as alkalization and or water treatment.

Furthermore, EP 0 328 019 discloses a method with which dietary cocoa edible fibers are provided from high-value starting materials for producing chocolate products rich in edible fiber.

For this, a cocoa powder with a low fat content or a pressed cake (cocoa liquor from which the cocoa butter has been removed) is treated enzymatically, the starch degradation products produced during the degradation of the starch are separated and the solid residue is washed and dried.

In the conventional production process of chocolate, about 15-20% (weight) waste of cocoa shells is produced.

It is a similar case with the residues of other plant foods such as the shells of other seeds and nuts, for example of rice, grain, beans and germs. These are likewise produced in a considerable amount during the conventional production process. They generally contain fats which oxidize and are therefore not suitable for consumption.

It is therefore the object to present a method for the further processing of plant remains with which the disadvantages of the known method are overcome and a valuable end product is provided.

The object is achieved by a method as claimed in claim 1. The method serves for the processing of plant remains, in particular shells of seeds and nuts, even more in particular of shells of cocoa beans, shells of grain seed and rice remains, remains of oil-containing germs and nuts. The plant remains are refined by the method according to the invention. In particular, dietary fibers and/or starting materials for a biogas plant are produced with the method.

Dietary fibers are understood here as meaning indigestible ballast substances suitable for foods.

The method according to the invention involves the steps (i) provision of plant remains with a shell fraction of at least 5% by weight, in particular 20% by weight, even more in particular at least 50% by weight, even more in particular at least 90% by weight, (ii) at least partial hydrolysis, in particular at least partial fermentation, of constituents of the plant remains, in particular at least partial hydrolysis of a carbohydrate, a fat and/or a protein and (iii) in particular separation of liquid phase with dissolved constituents and solid phase.

Preferably, the hydrolysis is accompanied by a fermentation in which, in an in particular anaerobic process, CO₂, CH₄, NH₃ and/or another gas (for example N₂, O₂, H₂S, H₂) is/are formed. In the present application, therefore, hydrolysis is always understood as meaning hydrolysis or hydrolysis and simultaneous fermentation.

Alternatively, the object is achieved by a method where (i) plant remains with a shell fraction of at least 5% by weight, in particular 20% by weight, even more in particular at least 50% by weight, even more in particular at least 90% by weight, are provided, and (ii) at least partial fermentation of constituents of the plant remains, in particular at least partial hydrolysis of a carbohydrate, a fat and/or a protein takes place. Then, separation of liquid phase with dissolved constituents and solid phase can take place.

The methods according to the invention go beyond a mere washing of the remains; in particular, a fermentation takes place with bacterial growth.

In an advantageous embodiment, the method according to the invention comprises a step in which it is checked whether a fermentation has actually taken place. For example, it is ascertained whether gas has formed, in particular whether CO₂, CH₄ and/or NH₃ has formed. For this purpose it can for example be measured whether the gas pressure during the process increases, whether certain gases/amounts of gases are formed, for example CO₂, CH₄, NH₃ and/or how the O₂ partial pressure in the liquid phase changes. Additionally or alternatively, the optical density (OD₆₀₀) of the liquid phase can be measured. This is a measure of the presence of microorganisms.

Alternatively or simultaneously, a detection reaction for the gases to be determined can be carried out.

In the present method, hydrolysis is understood in particular as being a process in which at least 3% by weight, preferably 5% by weight, even more preferably at least 10% by weight, even more preferably at least 30% by weight, of the proteins, fats and/or carbohydrates of the plant remains are degraded.

Likewise degraded by the method according to the invention are slimes which are strongly water-binding. Thus, cocoa shells comprise slimes which can absorb more than 400% of their own weight in water, which would have an adverse effect on the property, e.g. the rheology, of an end product.

Starting material for the method are plant remains which are generally produced as waste during a conventional production process, such as for example before or after the roasting of seed shells separated off from the cocoa beans, also called cocoa shells, remains of rice kernels, corn kernels, coffee beans, wheat or other grain kernels.

Since the valuable constituents cannot be separated completely from the remains in the conventional production process, the waste fraction also always comprises a certain percentage of the valuable fraction, i.e. of the core, the fruit flesh, the germ or the bean. This fraction can for example also be larger at the start or at the end of a production batch. According to the invention, the plant remains contain a shell fraction of at least 20% by weight, in particular at least 50% by weight, even more in particular at least 90% by weight.

During the hydrolysis, a cleavage of chemical constituents takes place as a result of the addition of a water molecule. During the fermentation, a degradation of organic materials takes place, e.g. sugars, proteins, fats and slimes, and/or a decomposition into smaller constituents, such as short-chain sugars, free amino acids, CO₂ and water.

In an aqueous solution, the in any case soluble constituents of the plant remains enter into solution, but so too do those constituents which only become soluble constituents as a result of the hydrolysis.

In a preferred embodiment of the method according to the invention, during step (ii) the fat fraction of the solid phase is reduced by at least 70%, compared to the fat fraction of the plant remains which are provided in step (i).

Alternatively or additionally, the ratio of water-insoluble to water-soluble constituents of the solid phase is increased by at least 20%, compared to the ratio in the plant remains which are provided in step (i).

The constituents which have gone into solution are preferably separated in the subsequent separation of liquid and solid phase from the remains.

The separation preferably takes place by filtration, by centrifugation, by decantation and/or by drying.

Preferably, the suspension is conveyed using a screw conveyor, an extruder or expeller, over a perforated plate, where the majority of the liquid runs off. Then, the mass is compacted under pressure, whereupon a residual moisture content of about 12% by weight is achieved.

Subsequently, the mass can be dried by heating and/or introduction of dry air such that a residual moisture content of 3-4% by weight is left behind. The resulting mass can be further processed; it can for example be easily ground.

The solid phase has significantly smaller fractions of water-soluble salts, fewer proteins, fewer fats and less cellulose than the plant remains which were starting material for the hydrolysis. Moreover, the fraction of slimes is reduced. The solid phase consists essentially of water-insoluble, i.e. largely indigestible fibers. The relatively taste-neutral, in the case of cocoa shells brown, solid phase, can be used as edible fiber. The solid phase is virtually no longer gellifying and exhibits improved grinding properties. Product shelf-life is increased since the basic materials responsible for spontaneous rotting and oxidation have already been degraded in the method according to the invention.

Harmful substances, such as pesticides and myotoxins, can likewise be significantly reduced by fermentation. Since an acidic medium is formed in step (ii), heavy metals can be better dissolved and washed out.

By adding nutrients, with the hydrolysis and subsequent removal of the water-soluble constituents it is possible to establish a ratio of the macronutrients (C:N:P:S, i.e. the quantitative ratio of carbon, nitrogen, phosphorus and sulfur) of 500:15:5:3, the C:N ratio is preferably between 10 and 45.

Preferably, for the hydrolysis and/or fermentation, a suspension of the plant remains in a solvent, in particular water, is prepared, in particular with a fraction of up to 40% by weight of dry mass.

The hydrolysis and/or fermentation preferably takes place in a tank that can be sealed, heated and supplied with pressure. Typically, 100-300 m³ of suspension can be processed in a tank.

The suspension is preferably mixed at a moderate stirring speed such that as homogeneous a mixing as possible is effected. The stirring ensures adequate mass transfer and prevents sedimentation. The longer the hydrolysis and/or fermentation process time, the slower the stirring speed can be. The more rapid the process is to take place, the higher the stirring speed must be.

The hydrolysis and/or fermentation is favored by suitable temperature and pressure conditions. Advantageously, the hydrolysis and/or fermentation takes place in a tank at a temperature between 25 and 40° C., in particular between 30 and 38° C., and at ambient pressure.

Alternatively, the hydrolysis, in particular the fermentation, takes place at a temperature between 45 and 60° C., in particular between 50 and 55° C., and at ambient pressure.

The hydrolysis and/or fermentation typically requires a period of up to 7 days, preferably of up to 5 days, even more preferably of up to 1-2 days, in particular of at least 3 hours.

The hydrolysis and/or fermentation is furthermore favored by the addition of enzymes, in particular of hydrolases such as lipases, amylases and proteases. Enzymes provide in particular for the catalytic hydrolysis of biomolecules, i.e. saccharides, proteins and fats, which are split into their building blocks.

Preferably, the hydrolysis and/or fermentation therefore takes place enzymatically.

Most hydrolyses and/or fermentations proceed more effectively and more quickly if the reaction takes place in an acidic or basic medium.

In an advantageous embodiment of the process according to the invention, the hydrolysis and/or fermentation takes place with the addition of an acid, in particular an organic acid such as acetic acid or formic acid or a base, in particular a phosphate buffer, a carbonate buffer, NaOH or KOH.

In this connection, in particular a pH between 3.0 and 6.5, preferably between 3.5 and 5.5, is set.

Typically, the pH during the hydrolysis and/or the fermentation drops from a value of about 7 to about 3.5.

The hydrolysis and/or fermentation can also proceed under the action of microorganisms. For this, on the one hand, the flora present on the material to be hydrolyzed can be used, or microorganisms capable of hydrolysis are added in a targeted manner. Preferably, microorganisms, in particular an inoculation bacterium for compost, are added for the hydrolysis in the method according to the invention, in particular in the amount 1/10 000, preferably 1/1000 (bacterium solution to suspension).

Some of the process product, in particular some of the liquid phase, can be used as inoculum for processing further starting materials.

In order to prepare the solid phase for use in the preparation of foods, the solids fraction is advantageously washed, sterilized and/or dried in a subsequent step.

The debacterization can take place in a roaster in which the thermal treatment simultaneously ensures sterilization and drying.

Preferably, the solids fraction is additionally ground. This can be performed before washing, drying and debacterization, before drying or even before washing.

The solids fraction can moreover additionally be colored, for example by alkalization.

Solid phase which is obtained from the hydrolysis and/or fermentation of cocoa shells, i.e. cocoa material rich in edible fiber, is suitable for supplementation during the manufacture of chocolates, compounds and/or fillings (e.g. dark chocolate and particularly for shaped chocolate and chocolate coating), cocoa drinks, confectionery bars, chocolate spread and bakery goods.

It is known that cellulose can barely be degraded at a pH of 7.5, which is optimum for methane formation. Consequently, fibrous biomass is generally firstly hydrolyzed and fermented and then the biogas fermentation is initiated.

The majority of the non-degradable fibers, in particular cellulose, has already been removed from the liquid phase which is formed in the method according to the invention. The liquid phase comprises predominantly ingredients in a form convertible for the methane-forming bacteria. It can therefore be used directly for producing biogas.

Advantageously, the method according to the invention therefore involves as a further process step the liquid phase, as an energy-rich liquid, being transferred to a biogas plant.

Moreover, the object is achieved through a use of the solid fraction from the process product of the method as described above as dietary fiber.

The object is moreover achieved by a use of the liquid fraction from the process product of the method as described above as feed for a biogas plant, in particular for generating electricity.

During the evolution of biogas, microorganisms utilize the chemical energy stored in carbohydrates, sugars, fats and proteins for their metabolism under anaerobic conditions (with the exclusion of air). The production of biogas is divided into four stages:

In the hydrolysis phase, fermentative bacteria cleave polymeric compounds such as proteins, fats and carbohydrates with the help of enzymes into simpler constituents (monomers) such as e.g. amino acids, glucose and fatty acid. As a rule, anaerobic bacteria are involved in this.

In the acid-forming phase, a fermentation and acid formation takes place. The dissolved substances are degraded by fermentative bacteria to give organic acids (acetic acid, propionic acid, butyric acid), lower alcohols, aldehydes, hydrogen, carbon dioxide and other gases such as ammonia and hydrogen sulfide. This procedure takes place until the bacteria are inhibited in their degradation process by their own degradation products (low pH).

The third, so-called acetogenic phase forms the binding member between the fermentation (acidification) and the methane formation. Here, the constituents are prepared by acetogenic bacteria in such a way that methanogenic bacteria can convert these into methane. The reaction proceeds endothermically, and heat therefore has to be supplied.

In the fourth phase, the methanogenesis, the acetic acid is cleaved by extremely oxygen-sensitive methanogenic bacteria to give methane, carbon dioxide and water. The water is removed from the biogas mixture during the condensation.

30% of the known methanogenic bacteria species use hydrogen and carbon dioxide for their metabolism and thus provide for a lower hydrogen partial pressure as a result of the reaction of hydrogen with the carbon dioxide formed beforehand. This is essential for the existence of the acetic acid bacteria although they themselves produce hydrogen. The methanogenic bacteria and the acetogenic bacteria thus live in symbiosis.

Following an interim buffering in a gas reservoir, the gas formed is generally used in a block-type thermoelectric power plant for producing electricity and/or heat. Alternatively, the gas produced can be used directly for combustion in a thermal process, for example during the heating of a roaster.

The gas output to be expected in the biogas plant is, per ton of cocoa shells, between 460 and 500 standard cubic meters (460-500 Nm³/t) with a methane concentration up to 60%±5%.

Moreover, the object on which the invention is based is achieved through a use of the liquid fraction from the process product of the method as described above as basic material or additive for a food, in particular an energy drink.

The liquid phase contains readily digestible constituents. It can be used after a sterilization immediately as basic material or supplement for a food, in particular a concentrated feed for animals or an energy drink.

The object on which the invention is based is moreover achieved by a method, in particular as described above, for processing plant remains, in particular shells of seeds and nuts, even more in particular shells of cocoa beans, shells of grain seed and rice remains, remains of oil-containing germs and nuts, where biogas is produced from at least some of the plant remains in a biogas plant and at least some of the biogas is used to provide energy for the production and/or processing of a product in which plant remains are left behind which, for their part, are processed in the method.

Preferably, the biogas is used directly in a thermal combustion process, preferably in a roaster.

In a preferred embodiment of the method, during the production and/or processing of the product, e.g. during the roasting of cocoa beans and/or during the production of cocoa nibs, no further energy is supplied apart from the energy provided by the biogas of the plant remains.

The method is thus an autonomous method for processing a plant product, in particular cocoa beans.

The method according to the invention takes place in a system which comprises
a production device, in particular a roaster or a roaster and a hydrolysis tank or a roaster and a fermentation reactor,
a biogas tank,
an input device, via which at least some of the plant remains can be transferred from the production device to the biogas tank and
a biogas processing facility in which energy can be obtained from the biogas obtained in the biogas tank for operating the production facility.

Optionally, a device for returning some of the plant remains introduced into the biogas tank back to the production device is provided.

The production device and the biogas tank are preferably spatially close to one another so that a pipeline or a belt can be used as input device.

The biogas processing facility can be, for example, an electricity generating device or a burner, depending on the form in which the energy is required during the production. The biogas processing facility can be integrated into the production device, for example in the form of a burner as part of a roaster.

The plant remains, which are produced for example in the roaster, can firstly be subjected, in particular as described above, in a hydrolysis tank to a hydrolysis, or in a fermentation reactor to a fermentation. Biogas is then obtained from the liquid phase in the biogas tank.

Alternatively, as early as during the hydrolysis and/or the fermentation, biogas can be formed which is preferably utilized in the biogas processing facility.

For this, the hydrolysis tank and/or the fermentation reactor and the biogas tank can form a spatial unit or the hydrolysis tank or the fermentation reactor is simultaneously configured as biogas tank.

The object is moreover achieved by a dietary fiber obtained from plant remains, in particular shells of seeds and nuts, even more in particular shells of cocoa beans, shells of grain seed and rice residues, residues of oil-containing germs and nuts, in particular in a method as described above, where the fiber has a fat fraction of less than 5% by weight, in particular less than 2.5% by weight, and/or a weight ratio of water-insoluble to water-soluble edible fibers of greater than 6.5.

### WORKING EXAMPLE

150 kg of cocoa shells with a content of about 1.5±1% by weight of fat, 4.5±2.5% by weight of water, 11±6% by weight of proteins, 23±7% by weight of cellulose and pentosans as well as 8±3% by weight of ash are suspended in 500 l of rainwater in a hydrolysis tank with a volume of 3 m³. Depending on how the cocoa shells were obtained and how much fruit fraction is still present, the fat content can also be significantly higher, e.g. up to 7% by weight.

The suspension is held at a temperature of about 25° C. or at 42° C. The mixture is stirred vigorously for one minute every four hours.

After an incubation time of 24 hours, solids and liquid are separated using a perforated plate with the exclusion of oxygen, where the liquid fraction is firstly fed into a buffer tank, which likewise has a volume of 3 m³.

The solid fraction is dried in the sun to a residual moisture content of 12% by weight. Then, the solid is pasteurized and thermally dried until it has a residual moisture of less than 5% by weight.

Grinding then takes place to a powder particle size at which 99.5% of the particles have a diameter of less than 75 μm.

The solid end product is a cocoa material rich in edible fiber. This cocoa material rich in edible fiber is particularly suitable for supplementation during the manufacture of chocolate compounds and so-called fillings (e.g. praline, confectionery and/or bread fillings), cocoa drinks, confectionery bars, chocolate spread and bakery goods.

As a result of the treatment, the fat fraction has been reduced by 70%. The ratio of water-insoluble to water-soluble edible fibers is about 5.5 for the starting material and greater than 19 for the end product.

The liquid phase is passed to a third biogas tank which has a volume of 40 m³ and in which there is 30 m³ of liquid. Excess liquid is then passed again to the first tank, to which again fresh cocoa shells are added and the hydrolysis begins. In this way, a closed liquid cycle is formed, which is advantageous particularly for plants in countries with little rainwater. Alternatively, fresh rainwater can always be used for the hydrolysis.

The temperature in the biogas tank is about 38-40° C. or 38-42° C. A biogas pressure of 2-5 bar is formed.

The biogas yield per ton of starting mass of cocoa shells (o DM, “organic dry matter”) is 485 standard cubic meters (485 Nm/t o DM±10%) with a methane concentration of 60% (±5%).

The biogas yield suffices to operate a roaster for cocoa beans in which the starting amount of cocoa shells is produced.

1-18. (canceled)

19. A method for processing plant remains, comprising the following method steps:
(i) provision of plant remains with a shell fraction of at least 20% by weight,
(ii) at least partial fermentation of constituents of the plant residues.

20. A method as claimed in claim 19, wherein the plant remains are shells of seeds and nuts.

21. A method as claimed in claim 20, wherein the plant remains are shells of cocoa beans, shells of grain seed and rice remains, residues of oil-containing germs and nuts.

22. A method as claimed in claim 19, comprising at least partial fermentation of a carbohydrate, a sugar, a fat and/or a protein.

23. A method as claimed in claim 19 comprising:
(i) provision of plant residues with a shell fraction of at least 20% by weight,
(ii) at least partial hydrolysis of constituents of the plant remains.

24. The method as claimed in claim 19 wherein during step (ii)
the fat fraction of the solid phase is reduced by at least 70%, based on the fat fraction of the plant remains, and/or
the weight ratio of water-insoluble to water-soluble constituents of the solid phase is increased by at least 20%, based on the ratio in the plant remains.

25. The method as claimed in claim 19, comprising:
separation of liquid phase with dissolved constituents and solid phase.

26. The method as claimed in claim 19, wherein for step (ii) a suspension of the plant remains in a solvent is prepared.

27. The method as claimed in claim 24, wherein the solvent is water.

28. The method as claimed in claim 24, wherein the suspension of the plant remains in the solvent has a fraction of up to 40% by weight dry mass.

29. The method as claimed in claim 19, wherein step (ii) takes place in a tank at a temperature between 25° C. and 55° C.

30. The method as claimed in claim 19, wherein step (ii) takes place enzymatically.

31. The method as claimed in claim 19, wherein step (ii) takes place at a pH between 3.0 and 6.5.

32. The method as claimed in claim 31, wherein step (ii) takes place with the addition of an acid or of a base.

33. The method as claimed in claim 32, wherein in the pH drops during step (ii).

34. The method as claimed in claim 19, wherein for step (ii) microorganisms are added.

35. The method as claimed in claim 25, wherein the solid phase is washed, sterilized and/or dried.

36. The method as claimed in claim 19, wherein the liquid phase is transferred as energy-rich liquid to a biogas plant.

37. The method as claimed in claim 19, wherein biogas that is formed in the method is transferred to a biogas processing facility.

38. The method as claimed in claim 19, comprising using the solid fraction from the process product of the method as dietary fiber.

39. The method as claimed in claim 19, comprising using the liquid fraction from the process product of the method as feed for a biogas plant.

40. The method as claimed in claim 39, wherein the biogas plant is for generating electricity.

41. The method as claimed in claim 19, comprising using the liquid fraction from the process product of the method as additive for an energy drink.

42. The method as claimed in claim 19, comprising:
(i) generation of biogas in a biogas plant from at least some of the plant remains,
(ii) use of at least some of the biogas for providing energy during the production and/or processing of a product in which plant remains are left over, which for their part are processed at least partially in the method.

43. A method for processing plant remains with the following steps:
(i) generation of biogas in a biogas plant from at least some of the plant remains,
(ii) use of at least some of the biogas for providing energy during the production and/or processing of a product in which plant remains are left over, which for their part are processed at least partially in the method.

44. A system for processing plant remains comprising
a production device, in particular a roaster or a roaster and a hydrolysis tank or a roaster and a fermentation reactor,
a biogas tank,
an input device, via which at least some of the plant remains can be transferred from the production device to the biogas tank and
a biogas processing facility in which energy can be obtained from the biogas obtained in the biogas tank for operating the production facility.

45. A dietary fiber obtained from plant remains
wherein
the fiber has a fat fraction of less than 5% by weight and/or has a weight ratio of water-insoluble to water-soluble edible fibers of greater than 6.5.

46. A method for processing plant residues comprising the following method steps:
(i) provision of plant residues with a shell fraction of at least 20% by weight,
(ii) at least partial hydrolysis of constituents of the plant remains.

47. The method as claimed in claim 46, comprising at least partial hydrolysis of a carbohydrate, a sugar, a fat and/or a protein.
